SEQUENCE LISTING (3 sequences)

SEQ ID NO: 1 - protein, 447 aa, Artificial Sequence
  M428L/N434S Fab-Fc Heavy Chain
EVQLVESGGGLVKPGGSLRLSCAASGFTFSSYTMHWVRQAPGKGLEWVSFISYDGNNKYYADSVKGRFTISRDNAKNSLYLQMNSLRAEDTAVYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 2 - protein, 480 aa, Artificial Sequence
  M428L/N434S scFv-Fc Heavy Chain
EIVLTQSPATLSASPGERVTLTCRASQSVGNDVAWYQQKPGQAPRLLINYASHRYTGVPDRFTGSGYGTEFTLTISSVQSEDFGVYYCQQDFSSPRTFGGGTKVEIKGKPGSGKPGSGKPGSGKPGSEVQLVESGGGLVKPGGSLRLSCVASGFTFSNYWMNWVRQAPGKGLEWVAEIRLYSNNYATHYAESVKGRFTISRDDSKSTLYLQMNNLKTEDTGVYYCTRYYGNYGGYFDVWGRGTLVTVSSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 3 - protein, 215 aa, Artificial Sequence
  M428L/N434S Light Chain
EIVLTQSPGTLSLSPGERATLSCRASQSVSSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2021/0079094 A1
(43) Pub. Date: Mar. 18, 2021

(54) DOSING OF A BISPECIFIC ANTIBODY THAT BINDS PD1 AND CTLA4

(71) Applicant: Xencor, Inc., Monrovia, CA (US)

(72) Inventors: Barbara Hickingbottom, Solana Beach, CA (US); Paul Foster, Rancho Santa Fe, CA (US)

(21) Appl. No.: 16/832,440

(22) Filed: Mar. 27, 2020

Related U.S. Application Data
(60) Provisional application No. 62/826,524, filed on Mar. 29, 2019.
(60) Provisional application No. 62/979,289, filed on Feb. 20, 2020.

(51) Int. Cl. C07K 16/28; A61P 35/00; A61K 9/00

(52) U.S. Cl. CPC C07K 16/2818; A61P 35/00; C07K 2317/515; C07K 2317/31; A61K 9/0019

(57) ABSTRACT

The present invention is directed to methods for treating a solid cancerous cancer through administering a bispecific anti-PD1 x anti-CTLA4 antibody.

### PRIORITY CLAIM

This application claims priority to U.S. Provisional Application Nos. 62/826,524, filed Mar. 29, 2019 and 62/979,289, filed Feb. 20, 2020, which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Mar. 26, 2020, is named 067461-5233-US_ST25.txt and is 10,469 bytes in size.

### BACKGROUND OF THE INVENTION

Since at least 1957 when Thomas and Burnet published their theory of immune surveillance, researchers in both immunology and oncology have been attempting to harness the power of the immune system to attack and possibly eliminate cancer (Decker et al, Cytokine Growth Factor Rev. 2009 August; 20(4):271-81). Published studies detail the ways in which cytotoxic lymphocytes, acting as custodians of cellular health, seek out and destroy aberrant somatic cells that have been transformed by spontaneous mutations, that is, cancer. Other published studies have elucidated the ways in which this process is regulated and influenced by other constituent cells of the immune system, by tumor cells, and by the overall tumor microenvironment.

These scientific inquiries led to the discovery of interleukin 2 (IL-2), first as a necessary element for T-cell maintenance in vivo, then as a promoter of T-cell proliferation in vivo, and ultimately as a therapeutic for metastatic renal cell carcinoma and melanoma (Morgan et al. Science. 1976; 193 (4257), 1007-1008; Rosenberg et al. NEJM. 1985; 313(23):1485-1492; U.S. Proleukin® (aldesleukin) Package Insert (Rev. July 2012). https://www.accessdata.fda.gov/drugsatfda_docs/label/2012/103293 s51301b1.pdf). The discovery and description of other cytokines and receptor-ligand interactions through which immune system and other cells exert effects led to the discovery of the powerful immune regulatory molecules, cytotoxic T lymphocyte antigen 4 (CTLA4) and programmed cell death protein 1 (PD1), which function in inflammatory environments to control and modulate the immune reaction. Further research showed that these and other checkpoint inhibitors, which largely determine the balance between T-cell activation and T-cell inhibition, were frequently co-opted by tumor cells to evade destruction by cytotoxic T cells (Postow et al., J Clin Oncol. 2015; 33(17):1974-1982).

CTLA4 is a coinhibitory receptor present on the surface of CD4+ and CD8+ T cells that is upregulated in inflammatory environments in which activated T cells are present. Its most prominent effect is to provide a physiological counterbalance to immune cell activation and thereby to control the intensity of the immune response. It exerts this effect by outcompeting CD28, a costimulatory molecule necessary for T-cell activation, for binding to CD80 and CD86 on antigen-presenting cells and tumor cells. The net effect of CTLA4 up-regulation is down-modulation of T-cell activation (Postow et al., J Clin Oncol. 2015; 33(17):1974-1982).

PD1 is an immunoregulatory molecule that is upregulated in the context of chronic and persistent antigen stimulation. In human subjects with cancer, PD1 is upregulated on the surface of activated tumor-infiltrating CD8+ T cells, as well as activated B cells and myeloid cells. Its primary ligands, PDL1 and PDL2, may be expressed on a wide range of cell types including antigen-presenting cells and tumor cells, and the overall effect of engagement of the ligands is to limit, terminate, or attenuate the cytotoxic and cytokine-producing capacity of cytotoxic T cells. This, in turn, results in an ineffective antitumor immune response and the persistence of tumors (Postow et al., J Clin Oncol. 2015; 33(17):1974-1982).

The discovery of CTLA4 and PD1 ultimately resulted in the first therapeutic checkpoint inhibitors, monoclonal antibodies designed to interact with these receptors, blocking their capacity to downregulate cytotoxic T-cell destruction of malignant cells. Blocking the down-regulation of T-cell activation, it was theorized, would result in reactivation of a dormant anti-tumor T-cell response. Several years later though researchers still disagree on precisely how anti-CTLA4 and anti-PD1 antibodies exert their effects, there is no longer doubt that the immune system, particularly cytotoxic T cells, can be reactivated to treat cancer. Beginning with the FDA approval of an anti-CTLA4 antibody, ipilimumab, for the treatment of advanced melanoma in 2011, the ensuing years have seen a long list of FDA approvals from the use of checkpoint immuno-oncology antibodies in a number of cancer indications. In addition to a supplemental approval for ipilimumab for melanoma in the adjuvant setting and in combination with nivolumab (anti-PD1) for advanced melanoma, there have been approvals for 5 monoclonal antibodies targeting PD1 and its ligands for the treatment of various solid cancerous tumors and hematological cancers, and hundreds of clinical trials are now being conducted with these or similar molecules in expanded indications (U.S. Yervoy® (ipilimumab) Package Insert (Revised: February 2018). https://www.accessdata.fda.gov/drugsatfda_docs/label/2018/125377 s0931b1.pdf; U.S. Opdivo® (nivolumab) Package Insert (revised on February 2018). https://www.accessdata.fda.gov/drugsatfda docs/label/2018/125554 s044s0451b1.pdf; U.S. Keytruda® (pembrolizumab) Package Insert (revised on November 2017). https://www.accessdata.fda.gov/drugsatfda_docs/label/2017/125514s0311b1.pdf; U.S. Tecentriq® (atelizumab) Package Insert (revised on April 2017). https://www.accessdata.fda.gov/drugsatfda_docs/label/2017/761034 s0011b1.pdf; U.S. Bavencio® (avelumab) Package Insert (revised on October 2017). https://www.accessdata.fda.govdrugsatfda_docs/label/2017/761049 s0021b1.pdf).

The surprise for many, even those in the field of immuno-oncology, has not been the success of checkpoint inhibitors in tumor types such as melanoma and renal cell carcinoma, which have long been considered immunogenic and immune-responsive; rather, it has been the effectiveness of checkpoint inhibitor therapy in tumor types that have previously been considered unlikely to respond to immunological approaches, such as non-small lung carcinoma and urothelial carcinoma. This success has resulted in an opportunity to offer benefit to millions of cancer subjects, but also a need for critical decision-making about the next steps in the development of checkpoint therapy, and the primary issue that now occupies the field is finding an efficient way to select and test the checkpoint therapy regimens that are most likely to be successful in specific cancer types and human subjects (Atkins et al., Semin Oncol. 2015 December; 42 Suppl 3:S12-9). This is necessary because of the expansion of oncology indications for which immunotherapy is considered at least testable, the multiple new checkpoint-directed therapies that are entering clinical development, and the discovery of many other biological elements that function in establishing and/or breaking immunotolerance toward cancer—all of which has created a deluge of possibilities. Thus, although the idea of reactivating the body's natural immune defenses against cancer has come to fruition in an entirely new modality of cancer treatment, the era of checkpoint immunotherapy has truly just begun, and the task now before the field is to expand the benefits of the therapy to as many cancer subjects as possible.

The facts that must be taken into account in planning the next stage are: 1) that checkpoint therapy has not benefited all cancer subjects; and 2) that such treatments are not without risk. In all the clinical trials published with checkpoint inhibitor therapies thus far, none has shown responses or other benefit in a majority of solid cancerous tumor subjects (Atkins et al., Semin Oncol. 2015 December; 42 Suppl 3:S12-9). In addition, although the toxicities of these drugs differ from those of cytotoxic chemotherapy, radiation therapy, molecularly targeted therapy, and even monoclonal antibodies targeted to other types of receptors, checkpoint inhibitors as a class carry a substantial risk of adverse events that may result in severe morbidity or death (Davies et al., Immunotargets Ther. 2017 Aug. 24; 6:51-71; Weber et al., J Clin Oncol. 2015 Jun. 20; 33(18):2092-9). And finally, except for a companion diagnostic for pembrolizumab use in some indications, a clinical biomarker that definitively predicts either response to checkpoint therapy or the development of toxicities has not yet been identified (Khagi et al., Cancer Metastasis Rev. 2017 March; 36(1):179-190). Assays for inflammatory gene signatures, mutational/neoantigen load, or PDL1 expression may permit enrichment of clinical trial subject populations for responders, but highly sensitive and specific assays that allow evidence-based human subject selection and de-selection for checkpoint therapy are not yet available. Thus, despite their positive impact on progression-free and/or overall survival in most oncology indications tested thus far, and despite their seemingly miraculous effects in a minority of human subjects who experience durable complete or almost complete responses, challenges remain in developing optimal regimens of the drugs and in matching treatments with the subjects most likely to benefit.

One approach to optimizing the benefit-risk ratio of checkpoint therapy is combination therapy. This idea has often been employed in oncology treatment to increase the initial response rate and prevent the loss of an initial response through the development of resistance and was proposed and explored as a way of rationalizing further development of checkpoint therapy by the Combination Immunotherapy Task Force of the Society for Immunotherapy of Cancer (Ott et al., J Immunother Cancer. 2017 Feb. 21; 5:16). According to the Task Force, the choice of combinations for further clinical testing among the almost overwhelming number of possible checkpoint combinations should be guided by the following principles: 1) given the widespread use and success of PD1-targeted therapies in multiple solid and hematological tumor types, as well as its acceptable tolerability, these drugs should be the backbone of any combinations; 2) treatments to be combined with PD1-directed therapy, including vaccines, adoptive T-cell therapies, molecularly targeted drugs, oncolytic viruses, or other checkpoint inhibitors, should work through a different pathway that is potentially synergistic with PD1 inhibition (Ott et al., J Immunother Cancer. 2017 Feb. 21; 5:16).

One combination of checkpoint therapies that work through different mechanisms to reactivate the immune response to cancer has already been tested in the clinic. The combination of ipilimumab (anti-CTLA4) and nivolumab (anti-PD1) was approved by the FDA for treatment of advanced melanoma, after demonstrating efficacy in a double-blind, randomized Phase 3 study comparing nivolumab alone (3 mg per kilogram of body weight every 2 weeks) to nivolumab (at a dose of 1 mg per kilogram) plus ipilimumab (at a dose of 3 mg per kilogram) every 3 weeks for 4 doses followed by nivolumab (3 mg per kilogram every 2 weeks), and ipilimumab alone (3 mg per kilogram every 3 weeks for 4 doses). The combination regimen resulted in a statistically significant increase in progression-free survival versus ipilimumab alone; 11.5 months for the combination versus 2.9 months for ipilimumab alone and 6.9 months for nivolumab alone. In addition, complete responses were reported in 22% of human subjects in the combination group, and none in the ipilimumab group. In this registrational clinical trial, median duration of response was not reached in either group; however, in data published subsequently for an expansion cohort treated at the dose and schedule now approved for human subjects with advanced melanoma, the median duration of response for the combination was 22.3 months, the 3-year overall survival rate was 63%, and the median overall survival had not been reached. Although these results were accompanied by significant toxicity, including an increased incidence of grade 3 and 4 adverse events in subjects receiving combination therapy, these events were not qualitatively different from those seen with single-agent checkpoint therapy, and there were no treatment-related deaths attributed to combination therapy in the registrational study, perhaps at least partly because clinical investigators and treating physicians have learned a great deal about the potential toxicities of checkpoint therapies and have created algorithms for diagnosis and treatment of these toxicities (U.S. Opdivo® (nivolumab) Package Insert (revised on February 2018). https://www.accessdata.fda.gov/drugsatfda_docs/label/2018/125554 s044s0451b1.pdf).

These results demonstrate that the combination of a CTLA4 inhibitor and a PD1 inhibitor can be effective in reactivating an anti-tumorresponse, although the additive effect of targeting these 2 checkpoint receptors has not been fully explained. Recently, however, one part of the explanation may have been provided in discovery of the special function that CTLA4 and PD1 double-positive cells play in the reestablishment of an immune response to cancer. In a study that measured the number of tumor-infiltrating T cells that co-expressed CTLA4 and PD1 at high levels, the number of such T cells was found to have a significant correlation with a subject's response to PD1 inhibitor treatment (Daud et al., J Clin Invest. 2016 Sep. 1; 126(9):3447-52). The presence of double checkpoint-positive T cells, which have been characterized as partially exhausted T cells, may thus be critical for successful reactivation of an antitumor response, and targeting such double-positive cells may result in an enhanced response.

While administering 2 separate checkpoint inhibitor antibodies in a single treatment regimen is one way to achieve the net effect of targeting and suppressing both checkpoint pathways, there are other, perhaps better, ways to accomplish dual checkpoint targeting. A bispecific monoclonal antibody such as XmAb20717, which binds both PD1 and CTLA4 receptors, should be able to block both pathways. Based on the demonstrated clinical effectiveness of combining inhibition of CTLA4 and PD1 and the pre-clinical evidence of XmAb20717's ability more specifically to target the CTLA4/PD1-double positive cells that appear critical to re-establishing an anti-tumor immune response, a compelling argument can be made that XmAb20717 should be tested in subjects with solid cancerous tumors.

Accordingly, there is a need for improved methods of delivering XmAb20717 to human subjects who possess solid cancerous tumors.

### BRIEF SUMMARY OF THE INVENTION

On one aspect, the invention provides a method for treating a solid cancerous tumor in a human subject, comprising administering to the human subject having the solid cancerous tumor an intravenous dose, once every 13-15 days, of between about 0.05 mg/kg and about 12 mg/kg of a bispecific antibody comprising a first monomer comprising SEQ ID NO: 1, a second monomer comprising SEQ ID NO: 2, and a light chain comprising SEQ ID NO: 3 for a time period sufficient to treat the solid cancerous tumor.

### BRIEF DESCRIPTION OF THE DRAWINGS

FIG. 1 depicts the structure of the antibody described herein. The XmAb20717 has a “bottle opener” format (also referred to as the “triple F” format). Bottle opener format antibodies include a) a first monomer that includes a first Fc domain and an scFv region, wherein the scFv includes a first variable heavy chain and a first variable light chain (also referred herein as a “scFv-Fc heavy chain;” b) a second monomer that includes a VH-CH1-hinge-CH2-CH3, wherein VH is a second variable heavy chain and CH2 and CH3 is a second Fc domain (also referred herein as a “Fab-Fc heavy chain;” and c) a light chain that includes a second variable light chain. As shown in FIG. 1, the scFv is the PD-1 binding domain and the second variable heavy chain and second variable light chain for the CTLA4 binding domain.

FIG. 2 depicts the amino acid sequences of the XmAb20717 antibody. The antibody is named using the Fab variable region first and the scFv variable region second, separated by a dash, followed by the chain designation (Fab-Fc heavy chain, scFv-Fc heavy chain or light chain). CDRs are underlined and slashes indicate the border(s) of the variable regions.

### DETAILED DESCRIPTION OF THE INVENTION

### I. Definitions

In order that the application may be more completely understood, several definitions are set forth below. Such definitions are meant to encompass grammatical equivalents.

By “CTLA4,” “CTLA-4,” “cytotoxic T-lymphocyte-associated protein 4,” “CD152,” or “cluster of differentiation 152” (e.g., Genebank Accession Number NP_001032720 (human isoform without transmembrane) and NP_005205 (human isoform with transmembrane)) as used herein is meant a coinhibitory receptor that is present on the surface of CD4+ and CD8+ T cells that is upregulated in inflammatory environments in which activated T cells are present. CTLA4 is a member of the immunoglobulin superfamily. CTLA4 contains an extracellular V domain, a transmembrane domain, and a cytoplasmic tail. Alternate splice variants, encoding different isoforms, have been characterized. The membrane-bound isoform functions as a homodimer interconnected by a disulfide bond, while the soluble isoform functions as a monomer. CTLA4 is capable of providing a physiological counterbalance to immune cell activation and thereby to control the intensity of the immune response. It exerts this effect by outcompeting CD28, a costimulatory molecule necessary for T-cell activation, for binding to CD80 and CD86 on antigen-presenting cells and tumor cells. The net effect of CTLA4 up-regulation is down-modulation of T-cell activation (Postow et al., J Clin Oncol. 2015; 33(17):1974-1982). CTLA4 can also inhibit T cell responses directly via SHP-2 and PP2A dephosphorylation of TCR-proximal signalling proteins such as CD3 and LAT. CTLA4 is also known to bind PI3K.

By “PD1,” “PD-1,” “Programmed cell death protein 1,” “CD279,” and “cluster of differentiation 279” (e.g., Genebank Accession Number NP_0015009 (human)) as used herein is meant a type I membrane protein that is a member of the extended CD28/CTLA-4 family of T cell regulators. PD1 includes an extracellular IgV domain followed by a transmembrane region and an intracellular tail PD1 is expressed on the surface of activated T cells, B cells and macrophage and is upregulated in the context of chronic and persistent antigen stimulation. In human subjects with cancer, PD1 is upregulated on the surface of activated tumor-infiltrating CD8+ T cells, as well as activated B cells and myeloid cells. Its primary ligands, PDL1 and PDL2, may be expressed on a wide range of cell types including antigen-presenting cells and tumor cells, and the overall effect of engagement of the ligands is to limit, terminate, or attenuate the cytotoxic and cytokine-producing capacity of cytotoxic T cells. This, in turn, results in an ineffective antitumor immune response and the persistence of tumors (Postow et al., J Clin Oncol. 2015; 33(17):1974-1982).

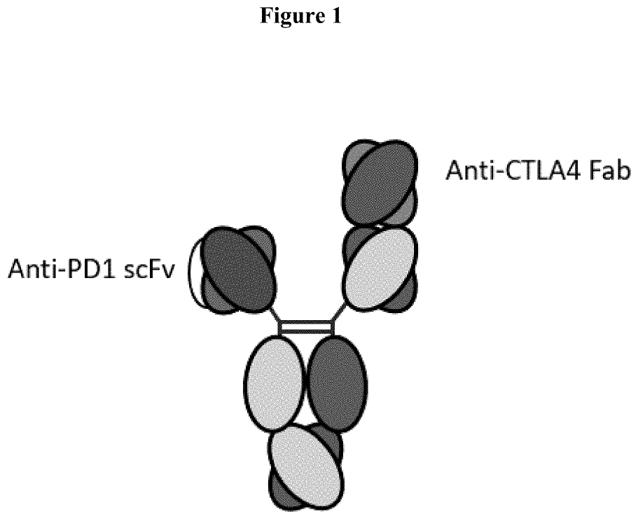

By “antigen binding domain” or “ABD” herein is meant a set of six Complementary Determining Regions (CDRs) that, when present as part of a polypeptide sequence, specifically binds a target antigen as discussed herein. Thus, a “checkpoint antigen binding domain” binds a target checkpoint antigen as outlined herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 for the heavy chain and vlCDR1, vlCDR2 and vlCDR3 for the light. The CDRs are present in the variable heavy and variable light domains, respectively, and together form an Fv region. (See Table 1 and related discussion above for CDR numbering schemes). Thus, in some cases, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used (e.g. from FIG. 1). In general, the C-terminus of the scFv domain is attached to the N-terminus of the hinge in the second monomer.

By “Fab” or “Fab region” as used herein is meant the polypeptide that comprises the VH, CH1, VL, and CL immunoglobulin domains, generally on two different polypeptide chains (e.g. VH-CH1 on one chain and VL-CL on the other). Fab may refer to this region in isolation, or this region in the context of a bispecific antibody of the invention. In the context of a Fab, the Fab comprises an Fv region in addition to the CH1 and CL domains.

By “Fv” or “Fv fragment” or “Fv region” as used herein is meant a polypeptide that comprises the VL and VH domains of an ABD. Fv regions can be formatted as both Fabs (as discussed above, generally two different polypeptides that also include the constant regions as outlined above) and scFvs, where the vl and vh domains are combined (generally with a linker as discussed herein) to form an scFv.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g. H.X_L.Y means N- to C-terminal is vh-linker-vl, and L.Y_H.X is vl-linker-vh.

By “Fc” or “Fc region” or “Fc domain” as used herein is meant the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus the definition of “Fc domain” includes both amino acids 231-447 (CH2-CH3) or 216-447 (hinge-CH2-CH3), or fragments thereof. An “Fc fragment” in this context may contain fewer amino acids from either or both of the N- and C-termini but still retains the ability to form a dimer with another Fc domain or Fc fragment as can be detected using standard methods, generally based on size (e.g. non-denaturing chromatography, size exclusion chromatography, etc.) Human IgG Fc domains are of particular use in the present invention, and can be the Fc domain from human IgG1, IgG2 or IgG4.

By “heavy chain constant region” herein is meant the CH1-hinge-CH2-CH3 portion of an antibody (or fragments thereof), excluding the variable heavy domain; in EU numbering of human IgG1 this is amino acids 118-447 By “heavy chain constant region fragment” herein is meant a heavy chain constant region that contains fewer amino acids from either or both of the N- and C-termini but still retains the ability to form a dimer with another heavy chain constant region.

By “target antigen” as used herein is meant the molecule that is bound specifically by the antigen binding domain comprising the variable regions of a given antibody. As discussed below, in the present case the target antigens are checkpoint inhibitor proteins.

By “target cell” as used herein is meant a cell that expresses a target antigen.

By “host cell” in the context of producing a bispecific antibody according to the invention herein is meant a cell that contains the exogeneous nucleic acids encoding the components of the bispecific antibody and is capable of expressing the bispecific antibody under suitable conditions. Suitable host cells are discussed below.

By “variable region” or “variable domain” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

The invention provides a number of antibody domains that have sequence identity to human antibody domains. Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc) are used. In one embodiment, sequence identity is done using the BLAST algorithm, using default parameters

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵M, at least about 10⁻⁶ M, at least about 10⁻⁷M, at least about 10−8 M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore, SPR or BLI assay.

As used herein, the IC₅₀ refers to an amount, concentration or dosage of a particular test compound that achieves a 50% inhibition of a maximal response, such as inhibition of the biological activity of PD1 and/or CTLA4, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

As used herein, the terms “treat,” “treatment” and “treating” refer to the reduction or amelioration or elimination of the progression, severity and/or effect associated with a solid cancerous tumor described herein, or the increase in the immune system response of the human subject, or the amelioration of one or more symptoms (preferably, one or more discernible symptoms) of a solid cancerous tumor described herein resulting from the administration of one or more therapies. In specific embodiments, the terms “treat,” “treatment” and “treating” refer to the amelioration of at least one measurable physical parameter of a solid cancerous tumor described herein, such as tumor size, rate of tumor growth, number of tumor cells, tumor invasiveness, presence of metastasis, or extent of metastasis. In other embodiments the terms “treat,” “treatment” and “treating” refer to the inhibition of the progression of a solid cancerous tumor described herein, either physically by, e.g., stabilization of a discernible symptom, physiologically by, e.g., stabilization of a physical parameter, or both. In other embodiments the terms “treat,” “treatment” and “treating” refer to an increase in the immune system response of the human subject, such as increased T cell infiltration, increased T cell activation, upregulation of IFN pathways, upregulation of antigen presentation pathway, increased presence of ICOS+CD4+ T cells following ipilimumab treatment, or increased Ki67+ induction in PD1 positive T cells following treatment with pembroluzumab or nivolumab).

### II. Overview

The invention provides methods of treating solid cancerous tumors through the administration of XmAb20717 according to a dosage regimen described herein.

### III. Antibodies

The present invention is directed to the administration of XmAb20717 for the treatment of particular solid cancerous tumors, as outlined herein and in U.S. patent application Ser. No. 15/623,314, US Publication No. 2018/0118836, U.S. Prov. Pat. App. Nos. 62/350,145, 62/355,511, and 62/420,500, all of which are expressly incorporated herein by reference, particularly for the bispecific formats of the figures, as well as all sequences, Figures and accompanying Legends therein.

In some embodiments, the bispecific anti-CTLA-4x anti-PD-1 antibodies have a “bottle opener” format (also referred to as the “triple F” format) as is generally depicted in FIG. 1. In this embodiment, the PD-1 antigen binding domain is the scFv in the bottle opener format and the CTLA-4 antigen binding domain is the Fab in the bottle opener format (terms as used in US Publication No. 20180118836 A1, all of which are expressly incorporated by reference in their entirety and specifically for all the definitions, sequences of CTLA-4 antigen binding domains and sequences of PD-1 antigen binding domains).

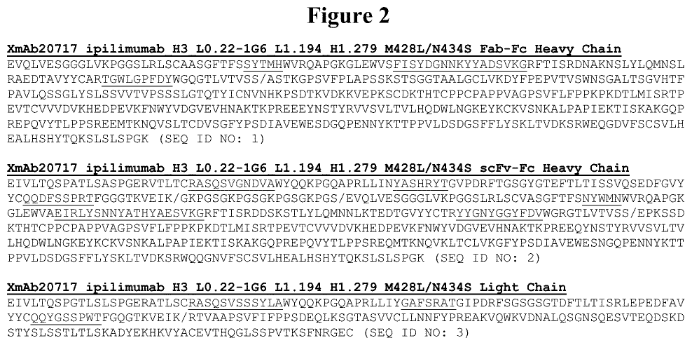

One bispecific antibody of particular use in the present invention, XmAb20717, is shown in FIG. 2. In certain embodiments, XmAb20717 includes a first monomer comprising SEQ ID NO: 1, a second monomer comprising SEQ ID NO: 2, and a light chain comprising SEQ ID NO: 3. XmAb20717 can be made as known in the art. In certain embodiments, XmAb20717 is made by expressing a nucleic acid composition that includes a) a first nucleic that encodes a first amino acid monomer comprising “Fab-Fc Heavy Chain;” b) a second nucleic that encodes a second amino acid monomer comprising “scFv-Fc Heavy Chain;” and c) a third nucleic that encodes a “light chain,” as depicted in FIG. 2. In some embodiments, the nucleic acids that encode for each of these three amino acid sequences can be incorporated into one or more expression vectors for expression.

As is known in the art, the nucleic acids encoding the components of the invention can be incorporated into expression vectors as is known in the art, and depending on the host cells used to produce XmAb20717. Generally, the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the invention are then transformed into any number of different types of host cells as is well known in the art, including mammalian, bacterial, yeast, insect and/or fungal cells, with mammalian cells (e.g., CHO cells), finding use in many embodiments.

In some embodiments, nucleic acids encoding each monomer and the optional nucleic acid encoding a light chain, as applicable depending on the format, are each contained within a single expression vector, generally under different or the same promoter controls. In embodiments of particular use in the present invention, each of these two or three nucleic acids are contained on a different expression vector.

XmAb20717 can be made by culturing host cells comprising the expression vector(s) as is well known in the art. Once produced, traditional antibody purification steps are done, including an ion exchange chromatography step. As discussed in U.S. patent application Ser. No. 15/623,314 and US Publication No. 2018/0118836, hereby incorporated by reference in their entirety and particularly for the discussions concerning purification, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “triple F” heterodimer (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

Once made, XmAb20717 can be administered to human subjects according to a dosage regimen described herein.

III. a) Pharmaceutical Compositions and Pharmaceutical Administration

XmAb20717 can be incorporated into pharmaceutical compositions suitable for administration to a human subject according to a dosage regimen described herein. Typically, the pharmaceutical composition comprises XmAb20717 and a pharmaceutically acceptable carrier. As used herein, “pharmaceutically acceptable carrier” includes any and all solvents, dispersion media, coatings, isotonic and absorption delaying agents, and the like that are physiologically compatible and are suitable for administration to a subject for the methods described herein. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as surfactants (such as nonionic surfactants) wetting or emulsifying agents (such as a polysorbate), preservatives or buffers (such as an organic acid, which as a citrate or an acetate), which enhance the shelf life or effectiveness of XmAb20717. Examples of pharmaceutically acceptable carriers include polysorbates (polysorbate-80).

In an exemplary embodiment, the pharmaceutical composition comprises XmAb20717, and a preservative or buffer. In an exemplary embodiment, the pharmaceutical composition comprises XmAb20717, and histidine. In an exemplary embodiment, the pharmaceutical composition comprises XmAb20717, and an acetate. In an exemplary embodiment, the pharmaceutical composition comprises XmAb20717, and sodium acetate. In an exemplary embodiment, the pharmaceutical composition comprises XmAb20717 and a citrate. In an exemplary embodiment, the pharmaceutical composition comprises XmAb20717 and sodium citrate.

In an exemplary embodiment, the pharmaceutical composition comprises XmAb20717 and an isotonic agent. In an exemplary embodiment, the pharmaceutical composition comprises XmAb20717 and a polyalcohol. In an exemplary embodiment, the pharmaceutical composition comprises XmAb20717 and mannitol. In an exemplary embodiment, the pharmaceutical composition comprises XmAb20717 and sorbitol. In an exemplary embodiment, the pharmaceutical composition comprises XmAb20717 and sodium chloride. In an exemplary embodiment, the pharmaceutical composition comprises XmAb20717 and potassium chloride.

In an exemplary embodiment, the pharmaceutical composition comprises XmAb20717 and a wetting or emulsifying agent. In an exemplary embodiment, the pharmaceutical composition comprises XmAb20717 and a polysorbate. In an exemplary embodiment, the pharmaceutical composition comprises XmAb20717 and polysorbate-80.

In an exemplary embodiment, the pharmaceutical composition comprises XmAb20717 and an intravenous solution stabilizer. In an exemplary embodiment, the intravenous solution stabilizer comprises a polysorbate and a citrate. In an exemplary embodiment, the pharmaceutical composition comprises XmAb20717 and sodium citrate and polysorbate-80.

In an exemplary embodiment, the pharmaceutical composition comprises XmAb20717 and a buffer and an isotonic agent. In an exemplary embodiment, the pharmaceutical composition comprises XmAb20717 and a buffer and sorbitol. In an exemplary embodiment, the pharmaceutical composition comprises XmAb20717 and an acetate and an isotonic agent. In an exemplary embodiment, the pharmaceutical composition comprises XmAb20717 and histidine and an isotonic agent. In an exemplary embodiment, the pharmaceutical composition comprises XmAb20717 and an acetate and sorbitol. In an exemplary embodiment, the pharmaceutical composition comprises XmAb20717 and sodium acetate and sorbitol. In an exemplary embodiment, the pharmaceutical composition comprises XmAb20717 and histidine and sorbitol.

In an exemplary embodiment, the pharmaceutical composition comprises XmAb20717 and a buffer and an isotonic agent and an intravenous solution stabilizer. In an exemplary embodiment, the pharmaceutical composition comprises XmAb20717 and a buffer and sorbitol and an intravenous solution stabilizer. In an exemplary embodiment, the pharmaceutical composition comprises XmAb20717 and an acetate and an isotonic agent and an intravenous solution stabilizer. In an exemplary embodiment, the pharmaceutical composition comprises XmAb20717 and histidine and an isotonic agent and an intravenous solution stabilizer. In an exemplary embodiment, the pharmaceutical composition comprises XmAb20717 and an acetate and sorbitol and an intravenous solution stabilizer. In an exemplary embodiment, the pharmaceutical composition comprises XmAb20717 and sodium acetate and sorbitol and an intravenous solution stabilizer. In an exemplary embodiment, the pharmaceutical composition comprises XmAb20717 and histidine and sorbitol and an intravenous solution stabilizer.

In an exemplary embodiment, the pharmaceutical composition comprises XmAb20717 and sodium chloride. In an exemplary embodiment, the pharmaceutical composition comprises XmAb20717 and sodium chloride and polysorbate-80. In an exemplary embodiment, the pharmaceutical composition comprises XmAb20717 and sodium citrate and sodium chloride. In an exemplary embodiment, the pharmaceutical composition comprises XmAb20717 and sodium citrate, sodium chloride, and polysorbate-80. In an exemplary embodiment, the pharmaceutical composition comprises XmAb20717 and sodium citrate, sodium chloride, sodium acetate, sorbitol and polysorbate-80. In an exemplary embodiment, the pharmaceutical composition comprises XmAb20717 and sodium citrate, sodium chloride, histidine, sorbitol and polysorbate-80.

The pharmaceutical compositions of this invention may be in a variety of forms. These include, for example, liquid, semi-solid and solid dosage forms, such as liquid solutions (e.g., injectable and infusible solutions), dispersions or suspensions. The form depends on the intended mode of administration and therapeutic application. Exemplary compositions are in the form of injectable or infusible solutions, such as compositions similar to those used for passive immunization of humans with other antibodies. In an exemplary embodiment, the mode of administration is intravenous. In an exemplary embodiment, the antibody is administered by intravenous infusion or injection.

Pharmaceutical compositions typically must be sterile and stable under the conditions of manufacture and storage. Sterile injectable solutions can be prepared by incorporating the antibody in the required amount in an appropriate solvent with one or a combination of ingredients enumerated herein, as required, followed by filtered sterilization. Generally, dispersions are prepared by incorporating the antibody into a sterile vehicle that contains a basic dispersion medium and the required other ingredients from those enumerated herein.

XmAb20717 can be administered by a variety of methods known in the art. In an exemplary embodiment, the route/mode of administration is intravenous injection. As will be appreciated by the skilled artisan, the route and/or mode of administration will vary depending upon the desired results.

### IV. Methods of Treating Solid Cancerous Tumors

The compositions of the invention can be used to treat certain solid cancerous tumors. In an exemplary embodiment, a composition of the invention is administered according to a method of the invention to treat a solid cancerous tumor. In an exemplary embodiment, the solid cancerous tumor is receptive to treatment by an antibody which binds to PD1. In an exemplary embodiment, the solid cancerous tumor is receptive to treatment by an antibody which binds to CTLA4. In an exemplary embodiment, the solid cancerous tumor is receptive to treatment by an antibody which binds to PD1 and CTLA4.

In an exemplary embodiment, the solid cancerous tumor is melanoma. In an exemplary embodiment, the solid cancerous tumor is melanoma, excluding uveal melanoma. In an exemplary embodiment, the solid cancerous tumor is cervical cancer. In an exemplary embodiment, the solid cancerous tumor is cervical carcinoma. In an exemplary embodiment, the solid cancerous tumor is breast carcinoma. In an exemplary embodiment, the solid cancerous tumor is breast carcinoma that is estrogen receptor, progesterone receptor, and human epidermal growth factor receptor 2 (HER2) negative (triple negative breast cancer [TNBC]). In an exemplary embodiment, the solid cancerous tumor is hepatocellular cancer. In an exemplary embodiment, the solid cancerous tumor is hepatocellular carcinoma. In an exemplary embodiment, the solid cancerous tumor is urothelial cancer. In an exemplary embodiment, the solid cancerous tumor is urothelial carcinoma. In an exemplary embodiment, the solid cancerous tumor is bladder cancer. In an exemplary embodiment, the solid cancerous tumor is head and neck cancer. In an exemplary embodiment, the solid cancerous tumor is squamous cell carcinoma of the head and neck. In an exemplary embodiment, the solid cancerous tumor is renal cell cancer. In an exemplary embodiment, the solid cancerous tumor is renal cell carcinoma. In an exemplary embodiment, the solid cancerous tumor is clear cell predominant type renal cell carcinoma.

In an exemplary embodiment, the solid cancerous tumor is colorectal cancer. In an exemplary embodiment, the solid cancerous tumor is MSI-high colorectal cancer. In an exemplary embodiment, the solid cancerous tumor is colorectal carcinoma. In an exemplary embodiment, the solid cancerous tumor is high microsatellite instability colorectal carcinoma. In an exemplary embodiment, the solid cancerous tumor is mismatch repair deficient colorectal carcinoma. In an exemplary embodiment, the solid cancerous tumor is endometrial cancer. In an exemplary embodiment, the solid cancerous tumor is MSI-high endometrial cancer. In an exemplary embodiment, the solid cancerous tumor is endometrial carcinoma. In an exemplary embodiment, the solid cancerous tumor is high microsatellite instability endometrial carcinoma. In an exemplary embodiment, the solid cancerous tumor is mismatch repair deficient endometrial carcinoma. In an exemplary embodiment, the solid cancerous tumor is small cell lung cancer. In an exemplary embodiment, the solid cancerous tumor is small cell lung carcinoma. In an exemplary embodiment, the solid cancerous tumor is non-small cell lung carcinoma. In an exemplary embodiment, the solid cancerous tumor is non-small cell lung cancer. In an exemplary embodiment, the solid cancerous tumor is gastric cancer. In an exemplary embodiment, the solid cancerous tumor is gastric adenocarcinoma. In an exemplary embodiment, the solid cancerous tumor is gastroesophageal junction cancer. In an exemplary embodiment, the solid cancerous tumor is gastroesophageal junction adenocarcinoma.

In an exemplary embodiment, the solid cancerous tumor is a microsatellite instability-high cancer. In an exemplary embodiment, the solid cancerous tumor is a mismatch repair deficient cancer.

In an exemplary embodiment, the solid cancerous tumor is mesothelioma. In an exemplary embodiment, the solid cancerous tumor is neuroendocrine cancer. In an exemplary embodiment, the solid cancerous tumor is high-grade neuroendocrine cancer. In an exemplary embodiment, the solid cancerous tumor is neuroendocrine carcinoma. In an exemplary embodiment, the solid cancerous tumor is anal cancer. In an exemplary embodiment, the solid cancerous tumor is anal carcinoma. In an exemplary embodiment, the solid cancerous tumor is squamous cell carcinoma of the anus.

In an exemplary embodiment, the solid cancerous tumor is prostate cancer. In an exemplary embodiment, the solid cancerous tumor is castration-resistant prostate carcinoma. In an exemplary embodiment, the solid cancerous tumor is nasopharyngeal cancer. In an exemplary embodiment, the solid cancerous tumor is nasopharyngeal carcinoma. In an exemplary embodiment, the solid cancerous tumor is Cholangiocarcinoma. In an exemplary embodiment, the solid cancerous tumor is basal cell cancer. In an exemplary embodiment, the solid cancerous tumor is basal cell skin cancer. In an exemplary embodiment, the solid cancerous tumor is basal cell carcinoma. In an exemplary embodiment, the solid cancerous tumor is ovarian cancer. In an exemplary embodiment, the solid cancerous tumor is ovarian carcinoma. In an exemplary embodiment, the solid cancerous tumor is fallopian tube cancer. In an exemplary embodiment, the solid cancerous tumor is fallopian tube carcinoma.

In an exemplary embodiment, the solid cancerous tumor is thymus cancer. In an exemplary embodiment, the solid cancerous tumor is thymoma. In an exemplary embodiment, the solid cancerous tumor is thymic carcinoma. In an exemplary embodiment, the solid cancerous tumor is penile cancer. In an exemplary embodiment, the solid cancerous tumor is Squamous Cell Carcinoma of the Penis. In an exemplary embodiment, the solid cancerous tumor is vulvar cancer. In an exemplary embodiment, the solid cancerous tumor is vulvar carcinoma. In an exemplary embodiment, the solid cancerous tumor is solid tumors with published evidence of anti-tumor activity with anti-PD1/PDL1 and/or anti-CTLA4-directed therapy. In an exemplary embodiment, the solid cancerous tumor is malignant adnexal tumor. In an exemplary embodiment, the solid cancerous tumor is malignant adnexal neoplasm. In an exemplary embodiment, the solid cancerous tumor is salivary gland cancer. In an exemplary embodiment, the solid cancerous tumor is non-squamous cell salivary gland carcinoma. In an exemplary embodiment, the solid cancerous tumor is bile duct cancer. In an exemplary embodiment, the solid cancerous tumor is bile duct carcinoma.

In an exemplary embodiment, the solid cancerous tumor described herein in a primary tumor. In an exemplary embodiment, the solid cancerous tumor described herein in a metastatic tumor.

Biological and Biochemical Functionality of the Heterodimeric Checkpoint Antibodies

Generally XmAb20717 is administered to human subjects with certain solid cancerous tumors, and efficacy is assessed in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays. For example, evaluation of changes in immune status (e.g. presence of ICOS+CD4+ T cells following ipilimumab treatment) along with “old fashioned” measurements such as tumor burden, size, invasiveness, LN involvement, metastasis, etc. can be done. Thus, any or all of the following can be evaluated: the inhibitory effects of the checkpoints on CD4+ T cell activation or proliferation, CD8+T (CTL) cell activation or proliferation, CD8+ T cell-mediated cytotoxic activity and/or CTL mediated cell depletion, NK cell activity and NK mediated cell depletion, the potentiating effects of the checkpoints on Treg cell differentiation and proliferation and Treg- or myeloid derived suppressor cell (MDSC)-mediated immunosuppression or immune tolerance, and/or the effects of the checkpoints on proinflammatory cytokine production by immune cells, e.g., IL-2, IFN-γ or TNF-α production by T or other immune cells.

In some embodiments, assessment of treatment is done by evaluating immune cell proliferation, using for example, CFSE dilution method, Ki67 intracellular staining of immune effector cells, and 3H-Thymidine incorporation method.

In some embodiments, assessment of treatment is done by evaluating the increase in gene expression or increased protein levels of activation-associated markers, including one or more of: CD25, CD69, CD137, ICOS, PD1, GITR, OX40, and cell degranulation measured by surface expression of CD107A.

In general, gene expression assays are done as is known in the art.

In general, protein expression measurements are also similarly done as is known in the art.

In some embodiments, assessment of treatment is done by assessing cytotoxic activity measured by target cell viability detection via estimating numerous cell parameters such as enzyme activity (including protease activity), cell membrane permeability, cell adherence, ATP production, co-enzyme production, and nucleotide uptake activity. Specific examples of these assays include, but are not limited to, Trypan Blue or PI staining, 51Cr or 35S release method, LDH activity, MTT and/or WST assays, Calcein-AM assay, Luminescent based assay, and others.

In some embodiments, assessment of treatment is done by assessing T cell activity measured by cytokine production, measure either intracellularly in culture supernatant using cytokines including, but not limited to, IFNγ, TNFα, GM-CSF, IL2, IL6, IL4, IL5, IL10, IL13 using well known techniques.

Accordingly, assessment of treatment can be done using assays that evaluate one or more of the following: (i) increases in immune response, (ii) increases in activation of αβ and/or γδ T cells, (iii) increases in cytotoxic T cell activity, (iv) increases in NK and/or NKT cell activity, (v) alleviation of αβ and/or γδ T-cell suppression, (vi) increases in pro-inflammatory cytokine secretion, (vii) increases in IL-2 secretion; (viii) increases in interferon-γ production, (ix) increases in Th1 response, (x) decreases in Th2 response, (xi) decreases or eliminates cell number and/or activity of at least one of regulatory T cells (Tregs).

Assays to Measure Efficacy

In some embodiments, T cell activation is assessed using a Mixed Lymphocyte Reaction (MLR) assay as is known in the art. An increase in activity indicates immunostimulatory activity. Appropriate increases in activity are outlined herein.

In one embodiment, the signaling pathway assay measures increases or decreases in immune response as measured for example by phosphorylation or de-phosphorylation of different factors, or by measuring other post translational modifications. An increase in activity indicates immunostimulatory activity. Appropriate increases in activity are outlined herein.

In one embodiment, the signaling pathway assay measures increases or decreases in activation of αβ and/or γδ T cells as measured for example by cytokine secretion or by proliferation or by changes in expression of activation markers like for example CD137, CD107a, PD1, etc. An increase in activity indicates immunostimulatory activity. Appropriate increases in activity are outlined herein.

In one embodiment, the signaling pathway assay measures increases or decreases in cytotoxic T cell activity as measured for example by direct killing of target cells like for example cancer cells or by cytokine secretion or by proliferation or by changes in expression of activation markers like for example CD137, CD107a, PD1, etc. An increase in activity indicates immunostimulatory activity. Appropriate increases in activity are outlined herein.

In one embodiment, the signaling pathway assay measures increases or decreases in NK and/or NKT cell activity as measured for example by direct killing of target cells like for example cancer cells or by cytokine secretion or by changes in expression of activation markers like for example CD107a, etc. An increase in activity indicates immunostimulatory activity. Appropriate increases in activity are outlined herein.

In one embodiment, the signaling pathway assay measures increases or decreases in αβ and/or γδ T-cell suppression, as measured for example by cytokine secretion or by proliferation or by changes in expression of activation markers like for example CD137, CD107a, PD1, etc. An increase in activity indicates immunostimulatory activity. Appropriate increases in activity are outlined herein.

In one embodiment, the signaling pathway assay measures increases or decreases in pro-inflammatory cytokine secretion as measured for example by ELISA or by Luminex or by Multiplex bead based methods or by intracellular staining and FACS analysis or by Alispot etc. An increase in activity indicates immunostimulatory activity. Appropriate increases in activity are outlined herein.

In one embodiment, the signaling pathway assay measures increases or decreases in IL-2 secretion as measured for example by ELISA or by Luminex or by Multiplex bead based methods or by intracellular staining and FACS analysis or by Alispot etc. An increase in activity indicates immunostimulatory activity. Appropriate increases in activity are outlined herein.

In one embodiment, the signaling pathway assay measures increases or decreases in interferon-γ production as measured for example by ELISA or by Luminex or by Multiplex bead based methods or by intracellular staining and FACS analysis or by Alispot etc. An increase in activity indicates immunostimulatory activity. Appropriate increases in activity are outlined herein.

In one embodiment, the signaling pathway assay measures increases or decreases in Th1 response as measured for example by cytokine secretion or by changes in expression of activation markers. An increase in activity indicates immunostimulatory activity. Appropriate increases in activity are outlined herein.

In one embodiment, the signaling pathway assay measures increases or decreases in Th2 response as measured for example by cytokine secretion or by changes in expression of activation markers. An increase in activity indicates immunostimulatory activity. Appropriate increases in activity are outlined herein.

In one embodiment, the signaling pathway assay measures increases or decreases cell number and/or activity of at least one of regulatory T cells (Tregs), as measured for example by flow cytometry or by IHC. A decrease in response indicates immunostimulatory activity. Appropriate decreases are the same as for increases, outlined herein.

In one embodiment, the signaling pathway assay measures increases or decreases in M2 macrophages cell numbers, as measured for example by flow cytometry or by IHC. A decrease in response indicates immunostimulatory activity. Appropriate decreases are the same as for increases, outlined herein.

In one embodiment, the signaling pathway assay measures increases or decreases in M2 macrophage pro-tumorigenic activity, as measured for example by cytokine secretion or by changes in expression of activation markers. A decrease in response indicates immunostimulatory activity. Appropriate decreases are the same as for increases, outlined herein.

In one embodiment, the signaling pathway assay measures increases or decreases in N2 neutrophils increase, as measured for example by flow cytometry or by IHC. A decrease in response indicates immunostimulatory activity. Appropriate decreases are the same as for increases, outlined herein.

In one embodiment, the signaling pathway assay measures increases or decreases in N2 neutrophils pro-tumorigenic activity, as measured for example by cytokine secretion or by changes in expression of activation markers. A decrease in response indicates immunostimulatory activity. Appropriate decreases are the same as for increases, outlined herein.

In one embodiment, the signaling pathway assay measures increases or decreases in inhibition of T cell activation, as measured for example by cytokine secretion or by proliferation or by changes in expression of activation markers like for example CD137, CD107a, PD1, etc. An increase in activity indicates immunostimulatory activity. Appropriate increases in activity are outlined herein.

In one embodiment, the signaling pathway assay measures increases or decreases in inhibition of CTL activation as measured for example by direct killing of target cells like for example cancer cells or by cytokine secretion or by proliferation or by changes in expression of activation markers like for example CD137, CD107a, PD1, etc. An increase in activity indicates immunostimulatory activity. Appropriate increases in activity are outlined herein.

In one embodiment, the signaling pathway assay measures increases or decreases in αβ and/or γδ T cell exhaustion as measured for example by changes in expression of activation markers. A decrease in response indicates immunostimulatory activity. Appropriate decreases are the same as for increases, outlined herein.

In one embodiment, the signaling pathway assay measures increases or decreases αβ and/or γδ T cell response as measured for example by cytokine secretion or by proliferation or by changes in expression of activation markers like for example CD137, CD107a, PD1, etc. An increase in activity indicates immunostimulatory activity. Appropriate increases in activity are outlined herein.

In one embodiment, the signaling pathway assay measures increases or decreases in stimulation of antigen-specific memory responses as measured for example by cytokine secretion or by proliferation or by changes in expression of activation markers like for example CD45RA, CCR7 etc. An increase in activity indicates immunostimulatory activity. Appropriate increases in activity are outlined herein.

In one embodiment, the signaling pathway assay measures increases or decreases in apoptosis or lysis of cancer cells as measured for example by cytotoxicity assays such as for example MTT, Cr release, Calcine AM, or by flow cytometry based assays like for example CFSE dilution or propidium iodide staining etc. An increase in activity indicates immunostimulatory activity. Appropriate increases in activity are outlined herein.

In one embodiment, the signaling pathway assay measures increases or decreases in stimulation of cytotoxic or cytostatic effect on cancer cells. as measured for example by cytotoxicity assays such as for example MTT, Cr release, Calcine AM, or by flow cytometry based assays like for example CFSE dilution or propidium iodide staining etc. An increase in activity indicates immunostimulatory activity. Appropriate increases in activity are outlined herein.

In one embodiment, the signaling pathway assay measures increases or decreases direct killing of cancer cells as measured for example by cytotoxicity assays such as for example MTT, Cr release, Calcine AM, or by flow cytometry based assays like for example CFSE dilution or propidium iodide staining etc. An increase in activity indicates immunostimulatory activity. Appropriate increases in activity are outlined herein.

In one embodiment, the signaling pathway assay measures increases or decreases Th17 activity as measured for example by cytokine secretion or by proliferation or by changes in expression of activation markers. An increase in activity indicates immunostimulatory activity. Appropriate increases in activity are outlined herein.

In one embodiment, the signaling pathway assay measures increases or decreases in induction of complement dependent cytotoxicity and/or antibody dependent cell-mediated cytotoxicity, as measured for example by cytotoxicity assays such as for example MTT, Cr release, Calcine AM, or by flow cytometry based assays like for example CFSE dilution or propidium iodide staining etc. An increase in activity indicates immunostimulatory activity. Appropriate increases in activity are outlined herein.

In one embodiment, T cell activation is measured for example by direct killing of target cells like for example cancer cells or by cytokine secretion or by proliferation or by changes in expression of activation markers like for example CD137, CD107a, PD1, etc. For T-cells, increases in proliferation, cell surface markers of activation (e.g. CD25, CD69, CD137, PD1), cytotoxicity (ability to kill target cells), and cytokine production (e.g. IL-2, IL-4, IL-6, IFNγ, TNF-α, IL-10, IL-17A) would be indicative of immune modulation that would be consistent with enhanced killing of cancer cells.

In one embodiment, NK cell activation is measured for example by direct killing of target cells like for example cancer cells or by cytokine secretion or by changes in expression of activation markers like for example CD107a, etc. For NK cells, increases in proliferation, cytotoxicity (ability to kill target cells and increases CD107a, granzyme, and perforin expression), cytokine production (e.g. IFNγ and TNF), and cell surface receptor expression (e.g. CD25) would be indicative of immune modulation that would be consistent with enhanced killing of cancer cells.

In one embodiment, γδ T cell activation is measured for example by cytokine secretion or by proliferation or by changes in expression of activation markers.

In one embodiment, Th1 cell activation is measured for example by cytokine secretion or by changes in expression of activation markers.

Appropriate increases in activity or response (or decreases, as appropriate as outlined above), are increases of 10%, 20%, 30%, 40%, 50%, 60%, 70%, 80%, 90%, 95% or 98 to 99% percent over the signal in either a reference sample or in control samples, for example test samples that do not contain an antibody of the invention. Similarly, increases of at least one-, two-, three-, four- or five-fold as compared to reference or control samples show efficacy.

Treatments

Once made, the compositions of the invention find use in a number of solid cancerous tumor applications, generally by inhibiting the suppression of T cell activation (e.g. T cells are no longer suppressed).

Accordingly, XmAb20717 finds use in the treatment of these cancers.

Dosage Regimen

In some embodiments, the XmAb20717 is administered to the human subject according to a dosage regimen described herein. Dosage regimens are adjusted to provide the optimum desired response (e.g., a therapeutic response). The efficient dosages and the dosage regimens for XmAb20717 depend on the disease or condition to be treated and may be determined by the persons skilled in the art.

In an exemplary embodiment, the intravenous dose of XmAb20717 is administered once between about 12 and about 17 days. In an exemplary embodiment, the intravenous dose of XmAb20717 is administered once between about 13 and about 15 days. In an exemplary embodiment, the intravenous dose of XmAb20717 is administered once every 13-15 days. In an exemplary embodiment, the intravenous dose of XmAb20717 is administered once between about 12 and about 16 days. In an exemplary embodiment, the intravenous dose of XmAb20717 is administered once every 12-16 days. In an exemplary embodiment, the intravenous dose of XmAb20717 is administered once every 14-16 days. In an exemplary embodiment, the intravenous dose of XmAb20717 is administered once about every 14 days. In an exemplary embodiment, the intravenous dose of XmAb20717 is administered once every 14 days. In an exemplary embodiment, the intravenous dose of XmAb20717 is administered once about every two weeks. In an exemplary embodiment, the intravenous dose of XmAb20717 is administered once every two weeks. In an exemplary embodiment, the intravenous dose of XmAb20717 is administered once between about 13 and about 17 days. In an exemplary embodiment, the intravenous dose of XmAb20717 is administered once every 13-17 days. In an exemplary embodiment, the intravenous dose of XmAb20717 is administered once about every 15 days. In an exemplary embodiment, the intravenous dose of XmAb20717 is administered once every 15 days.

In an exemplary embodiment, the administering of the intravenous dose to the human subject lasts between about 45 minutes and about 75 minutes. In an exemplary embodiment, the administering of the intravenous dose to the human subject lasts between 45 minutes and 75 minutes. In an exemplary embodiment, the administering of the intravenous dose to the human subject lasts about one hour. In an exemplary embodiment, the administering of the intravenous dose to the human subject lasts one hour.

In an exemplary embodiment, the XmAb20717 is administered for a time period sufficient to treat the solid cancerous tumor. In an exemplary embodiment, the XmAb20717 is administered for a time period sufficient to maintain the treatment of the solid cancerous tumor. In an exemplary embodiment, the time period is between about 1 and about 9 weeks. In an exemplary embodiment, the time period is between about 2 and about 7 weeks. In an exemplary embodiment, the time period is between about 3 and about 9 weeks. In an exemplary embodiment, the time period is between about 1 and about 8 weeks. In an exemplary embodiment, the time period is between about 3 and about 5 weeks. In an exemplary embodiment, the time period is about 4 weeks. In an exemplary embodiment, the time period is 4 weeks. In an exemplary embodiment, the time period is between about 7 and about 9 weeks. In an exemplary embodiment, the time period is about 8 weeks. In an exemplary embodiment, the time period is 8 weeks. In an exemplary embodiment, the time period is from about 1 week to about 10 years. In an exemplary embodiment, the time period is from about 1 week to about 9.5 years. In an exemplary embodiment, the time period is from about 1 week to about 9 years. In an exemplary embodiment, the time period is from about 1 week to about 8.5 years. In an exemplary embodiment, the time period is from about 1 week to about 8 years. In an exemplary embodiment, the time period is from about 1 week to about 7.5 years. In an exemplary embodiment, the time period is from about 1 week to about 7 years. In an exemplary embodiment, the time period is from about 1 week to about 6.5 years. In an exemplary embodiment, the time period is from about 1 week to about 6 years. In an exemplary embodiment, the time period is from about 1 week to about 5.5 years. In an exemplary embodiment, the time period is from about 1 week to about 5 years. In an exemplary embodiment, the time period is from about 1 week to about 4.5 years. In an exemplary embodiment, the time period is from about 1 week to about 4 years. In an exemplary embodiment, the time period is from about 1 week to about 3.5 years. In an exemplary embodiment, the time period is from about 1 week to about 3 years. In an exemplary embodiment, the time period is from about 1 week to about 2.5 years. In an exemplary embodiment, the time period is from about 1 week to about 2 years. In an exemplary embodiment, the time period is from about 1 week to about 1.5 years. In an exemplary embodiment, the time period is from about 1 week to about 1 year. In an exemplary embodiment, the time period is from about 1 week to about 3 months, or about 4 months, or about 5 months, or about 6 months, or about 7 months, or about 8 months, or about 9 months, or about 10 months or about 11 months. In an exemplary embodiment, the time period is until a positive therapeutic response is achieved. In an exemplary embodiment, the time period is as long as a positive therapeutic response is maintained. In an exemplary embodiment, the time period is until a complete response is achieved. In an exemplary embodiment, the time period is as long as until a bone marrow transplant can be performed on the human subject.

In an exemplary embodiment, the XmAb20717 is administered once every 13-15 days for a time period lasting between about 1 and about 9 weeks. In an exemplary embodiment, the XmAb20717 is administered once every 13-15 days for a time period lasting between about 2 and about 7 weeks. In an exemplary embodiment, the XmAb20717 is administered once every 13-15 days for a time period lasting between about 3 and about 9 weeks. In an exemplary embodiment, the XmAb20717 is administered once every 13-15 days for a time period lasting between about 1 and about 8 weeks. In an exemplary embodiment, the XmAb20717 is administered once every 13-15 days for a time period lasting between about 3 and about 5 weeks. In an exemplary embodiment, the XmAb20717 is administered once every 13-15 days for a time period lasting about 4 weeks. In an exemplary embodiment, the XmAb20717 is administered once every 13-15 days for a time period lasting 4 weeks. In an exemplary embodiment, the XmAb20717 is administered once every 13-15 days for a time period lasting between about 7 and about 9 weeks. In an exemplary embodiment, the XmAb20717 is administered once every 13-15 days for a time period lasting about 8 weeks. In an exemplary embodiment, the XmAb20717 is administered once every 13-15 days for a time period lasting 8 weeks. In an exemplary embodiment, the XmAb20717 is administered once every 13-15 days for a time period lasting until a positive therapeutic response is achieved. In an exemplary embodiment, the XmAb20717 is administered once every 13-15 days for a time period sufficient to treat the solid cancerous tumor. In an exemplary embodiment, the XmAb20717 is administered once every 13-15 days for a time period sufficient to maintain the treatment of the solid cancerous tumor.

The dosage may be determined or adjusted by measuring the amount of XmAb20717 of the present invention in the blood upon administration using techniques known in the art, for instance taking out a biological sample and using anti-idiotypic antibodies which target the antigen binding region of the XmAb20717.

In an exemplary embodiment, the intravenous dose is between about 0.05 mg/kg and about 12 mg/kg. In an exemplary embodiment, the intravenous dose is between about 0.15 mg/kg and about 10.0 mg/kg.

In an exemplary embodiment, the intravenous dose is between about 0.05 mg/kg and about 0.25 mg/kg. In an exemplary embodiment, the intravenous dose is between about 0.07 mg/kg and about 0.23 mg/kg. In an exemplary embodiment, the intravenous dose is between about 0.09 mg/kg and about 0.21 mg/kg. In an exemplary embodiment, the intravenous dose is between about 0.11 mg/kg and about 0.19 mg/kg. In an exemplary embodiment, the intravenous dose is between about 0.13 mg/kg and about 0.17 mg/kg. In an exemplary embodiment, the intravenous dose is about 0.15 mg/kg. In an exemplary embodiment, the intravenous dose is 0.15 mg/kg.

In an exemplary embodiment, the intravenous dose is between about 0.2 mg/kg and about 0.4 mg/kg. In an exemplary embodiment, the intravenous dose is between about 0.22 mg/kg and about 0.38 mg/kg. In an exemplary embodiment, the intravenous dose is between about 0.24 mg/kg and about 0.36 mg/kg. In an exemplary embodiment, the intravenous dose is between about 0.26 mg/kg and about 0.34 mg/kg. In an exemplary embodiment, the intravenous dose is between about 0.28 mg/kg and about 0.32 mg/kg. In an exemplary embodiment, the intravenous dose is about 0.3 mg/kg. In an exemplary embodiment, the intravenous dose is 0.3 mg/kg.

In an exemplary embodiment, the intravenous dose is between about 0.5 mg/kg and about 1.5 mg/kg. In an exemplary embodiment, the intravenous dose is between about 0.6 mg/kg and about 1.4 mg/kg. In an exemplary embodiment, the intravenous dose is between about 0.7 mg/kg and about 1.3 mg/kg. In an exemplary embodiment, the intravenous dose is between about 0.8 mg/kg and about 1.2 mg/kg. In an exemplary embodiment, the intravenous dose is between about 0.9 mg/kg and about 1.1 mg/kg. In an exemplary embodiment, the intravenous dose is about 1.0 mg/kg. In an exemplary embodiment, the intravenous dose is 1.0 mg/kg.

In an exemplary embodiment, the intravenous dose is between about 1.0 mg/kg and about 5.0 mg/kg. In an exemplary embodiment, the intravenous dose is between about 1.5 mg/kg and about 4.5 mg/kg. In an exemplary embodiment, the intravenous dose is between about 2.0 mg/kg and about 4.0 mg/kg. In an exemplary embodiment, the intravenous dose is between about 1.0 mg/kg and about 3.0 mg/kg. In an exemplary embodiment, the intravenous dose is between about 3.0 mg/kg and about 5.0 mg/kg. In an exemplary embodiment, the intravenous dose is between about 2.2 mg/kg and about 3.8 mg/kg. In an exemplary embodiment, the intravenous dose is between about 2.4 mg/kg and about 3.6 mg/kg. In an exemplary embodiment, the intravenous dose is between about 2.6 mg/kg and about 3.4 mg/kg. In an exemplary embodiment, the intravenous dose is between about 2.8 mg/kg and about 3.2 mg/kg. In an exemplary embodiment, the intravenous dose is between about 2.9 mg/kg and about 3.1 mg/kg. In an exemplary embodiment, the intravenous dose is about 3.0 mg/kg. In an exemplary embodiment, the intravenous dose is 3.0 mg/kg.

In an exemplary embodiment, the intravenous dose is between about 3.0 mg/kg and about 8.5 mg/kg. In an exemplary embodiment, the intravenous dose is between about 3.5 mg/kg and about 8.0 mg/kg. In an exemplary embodiment, the intravenous dose is between about 4.0 mg/kg and about 7.5 mg/kg. In an exemplary embodiment, the intravenous dose is between about 4.5 mg/kg and about 7.0 mg/kg. In an exemplary embodiment, the intravenous dose is between about 3.0 mg/kg and about 6.0 mg/kg. In an exemplary embodiment, the intravenous dose is between about 5.0 mg/kg and about 8.5 mg/kg. In an exemplary embodiment, the intravenous dose is between about 4.0 mg/kg and about 8.0 mg/kg. In an exemplary embodiment, the intravenous dose is between about 4.2 mg/kg and about 7.8 mg/kg. In an exemplary embodiment, the intravenous dose is between about 4.4 mg/kg and about 7.6 mg/kg. In an exemplary embodiment, the intravenous dose is between about 4.6 mg/kg and about 7.4 mg/kg. In an exemplary embodiment, the intravenous dose is between about 4.8 mg/kg and about 7.2 mg/kg. In an exemplary embodiment, the intravenous dose is between about 5.0 mg/kg and about 7.0 mg/kg. In an exemplary embodiment, the intravenous dose is between about 5.2 mg/kg and about 6.8 mg/kg. In an exemplary embodiment, the intravenous dose is between about 5.4 mg/kg and about 6.6 mg/kg. In an exemplary embodiment, the intravenous dose is between about 5.6 mg/kg and about 6.4 mg/kg. In an exemplary embodiment, the intravenous dose is between about 5.8 mg/kg and about 6.2 mg/kg. In an exemplary embodiment, the intravenous dose is about 6.0 mg/kg. In an exemplary embodiment, the intravenous dose is 6.0 mg/kg.

In an exemplary embodiment, the intravenous dose is between about 8.0 mg/kg and about 12.0 mg/kg. In an exemplary embodiment, the intravenous dose is between about 8.3 mg/kg and about 11.7 mg/kg. In an exemplary embodiment, the intravenous dose is between about 8.6 mg/kg and about 11.4 mg/kg. In an exemplary embodiment, the intravenous dose is between about 8.9 mg/kg and about 11.1 mg/kg. In an exemplary embodiment, the intravenous dose is between about 9.2 mg/kg and about 10.8 mg/kg. In an exemplary embodiment, the intravenous dose is between about 9.5 mg/kg and about 10.5 mg/kg. In an exemplary embodiment, the intravenous dose is between about 9.8 mg/kg and about 10.2 mg/kg. In an exemplary embodiment, the intravenous dose is about 10.0 mg/kg. In an exemplary embodiment, the intravenous dose is 10.0 mg/kg.

In some embodiments, the XmAb20717 is administered intravenously. In some embodiments, the XmAb20717 is administered once-every-two-weeks until disease progression, unacceptable toxicity, or individual choice.

In some embodiments, the XmAb20717 is a front line therapy, second line therapy, third line therapy, fourth line therapy, fifth line therapy, or sixth line therapy.

In some embodiments, the XmAb20717 treats a refractory solid cancerous tumor. In some embodiments, the XmAb20717 is a maintenance therapy.

A medical professional having ordinary skill in the art may readily determine and prescribe the effective amount of the antibody composition required. For example, a physician could start doses of the medicament employed in the antibody composition at levels lower than that required in order to achieve the desired therapeutic effect and gradually increase the dosage until the desired effect is achieved.

Treatment Modalities

In the methods of the invention, therapy is used to provide a positive therapeutic response with respect to a disease or condition. By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased human subject survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling and counting of tumor cells in the circulation.

In addition to these positive therapeutic responses, the subject undergoing therapy may experience the beneficial effect of an improvement in the symptoms associated with the disease.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for XmAb20717 depend on the disease or condition to be treated and may be determined by the persons skilled in the art.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### EXAMPLES

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in US Publications 2015/0307629, and 2014/0288275, as well as PCT Publication WO2014/145806, as well as U.S. application Ser. Nos. 62/085,027, 14/952,714, and 15/141,350, all of which are expressly incorporated by reference in their entirety and particularly for the techniques outlined therein.

### Example 1

XmAb20717 Treatment Plan

This is a two-part Phase 1, multiple-dose, ascending-dose escalation study and expansion designed to define a Maximum Tolerated Dose and/or Recommended Dose (MTD/RD) and regimen, as well as preliminarily assessing potential anti-tumor activity of XmAb20717 in subjects with selected advanced solid cancerous tumors. All eligible subjects will have relapsed or refractory disease after standard therapy.

XmAb20717 is a humanized bsAb that binds both PD1 and CTLA4. The XmAb20717 pharmaceutical composition is a sterile liquid supplied in single-use glass vials. Each 10 mL vial is filled with 10.5 mL of pharmaceutical composition containing 10.0±1.0 mg/mL of XmAb20717, in 20 mM histidine, 250 mM sorbitol, and 0.01% (w/v) polysorbate-80 at pH 6.2. Each product vial is intended to deliver 10.0 mL of drug solution.

Prior to administration, XmAb20717 will be diluted to the final required concentration in an ethylene/polypropylene copolymer infusion bag containing 0.9% Sodium Chloride Injection, USP. After dilution, the bag containing XmAb20717 should be gently inverted 2 to 3 times to mix the solution. The bag must not be shaken.

XmAb20717 administration should begin as soon as possible after the dosing solution is made. If there is a delay in administration, the dosing solution may be stored at 2-8° C. for no more than 24 hours or at room temperature for no more than 4 hours prior to infusion. The full-calculated dose will be administered based on the subject's actual baseline weight measurement in kilograms. Following the first dose, subsequent doses will be modified only if the subject's weight changes by more than 10% from the Day−1 weight, at which point it will be recalculated using the current weight.

In Part A of the study, subjects will be enrolled into escalating dose cohorts to establish an MTD/RD(s) for a dosing regimen consisting of infusions on Days 1 and 15 of each 28-day cycle.

The decision to escalate to higher-dose cohorts will proceed according to the Dose Escalation Plan, and the final decision to escalate to a higher dose level will be based on review of the aggregate safety data for all subjects through Cycle 1 Day 28. For each escalation cohort, the first subject in the cohort will be dosed and observed for a minimum of 24 hours before study drug is administered to the remainder of the cohort. All subjects will be assessed for the development of dose-limiting toxicity (DLT) during treatment with XmAb20717. The assessment period is defined as: Cycle 1, Days 1 to 28.

Once the MTD/RD(s) and dosing regimen are established, Part B of the study will begin. In Part B, additional subjects with advanced melanoma (excluding uveal), renal cell carcinoma (clear cell predominant type), and non-small cell lung carcinoma will be enrolled into disease-specific expansion cohorts of up to 20 subjects each.

Dosing Schedule

Each subject was administered XmAb20717 IV at a constant infusion rate over 1 hour. The initial treatment period for each subject in this study was 2 cycles. Each cycle was 28 days long and consisted of 2 doses of XmAb20717, on Days 1 and 15. Six dose levels were planned for the dose-escalation phase of the study (Part A). Those dose levels are: 0.15, 0.3, 1.0, 3.0, 6.0, and 10.0 mg/kg. A subject's first dose was based on the Day−1 baseline weight in kilograms. Subsequent doses were modified only if the subject's weight changed by more than 10% from the Day−1 weight, at which point the dose was recalculated using the subject's current weight. That dose was continued for the remainder of the trial, unless there was a subsequent 10% weight change.

TABLE 1
Dose Level Scheme

| | Planned | Human |
|---|---|---|
| Cohort | Dose | Subjects |
| 1 | 0.15 mg/kg | 3 (+3) |
| 2 | 0.3 mg/kg | 3 (+3) |
| 3 | 1.0 mg/kg | 3 (+3) |
| 4 | 3.0 mg/kg | 3 (+3) |
| 5 | 6.0 mg/kg | 3 (+3) |
| 6 | 10.0 mg/kg | 3 (+3) |

A minimum of 3 subjects were enrolled in each dose-escalation cohort. No 2 subjects within a cohort started treatment with XmAb20717 on the same day; the first subject was dosed and observed for a minimum of 24 hours before study drug was administered to the remainder of the cohort. All subjects were assessed for the development of dose-limiting toxicities (DLT) during treatment with XmAb20717. If none of the first 3 subjects experienced a DLT during the period, escalation to the next dosing level occurred. If any of the first 3 subjects in a dosing cohort experienced a DLT during the period, the cohort was expanded to a total of 6 human subjects or until a second subject in the cohort experienced a DLT. If there were no additional DLTs, escalation to the next dose level occurred. If there are 1 or more additional DLTs, the MTD will have been exceeded, and the next lower dose level will be expanded to 6 subjects. If no more than 1 subject experiences a DLT at the de-escalated dose level, it will be the MTD.

TABLE 2
Dose Escalation Scheme
Standard Dose Escalation Phase

| Number | Number of | |
|---|---|---|
| of Human | Subjects Enrolled | |
| Subjects | and Assessable for | |
| with at | Safety Following | |
| Least | 2 Doses of | |
| One DLT | XmAb20717 | Escalation Decision |
| 0 | 3 | Escalate to the next |
| | | higher dose level |
| 1 | 3 | Enroll 3 additional human |
| | | subjects at the same dose level |
| 1 | 6 | Escalate to the next higher |
| | | dose level |
| 2 | Up to 3 or 6 | No dose escalation may occur; |
| | | The dose must be de-escalated. |

DLT = dose-limiting toxicity

Results: As of Feb. 5, 2020, 34 patients were treated in cohorts 1-6 at fixed doses of 0.15 to 10 mg/kg. Patients had a median age of 57 years (range 32-81), a median time since initial diagnosis of 42 months (range 3-313) and a median of 4 prior systemic therapies (range 0-9). 68% of patients had a TNM stage of III/IV and 68% had been exposed to checkpoint therapy.

XmAb20717 treatment was generally well-tolerated through the highest dose cohort tested. Overall rates of Gr3/4 immune-related AEs occurred in 8 (24%) patients including elevations of transaminases 3 (9%), rash 2 (6%), lipase and amylase 1 (3%, without clinical symptoms or radiographic evidence of pancreatitis), lipase (alone) 1 (3%), pruritus 1 (3%), hyperglycaemia 1 (3%), arthritis 1 (3%) and colitis 1 (3%), all reversible.

Responses were evaluated based on RECIST 1.1 criteria and there was 1 complete response reported (melanoma, progressed on prior pembrolizumab) at 10 mg/kg (highest dose level). Dose-dependent pharmacodynamic activity consistent with dual PD-1/CTLA-4 blockade was noted, namely a proliferative burst of both CD8 and CD4 T cells and induction of IFN-inducible chemokines (Table 3).

TABLE 3
Maximum change from baseline in the first two cycles

| | Dose | CD8 + | CD4 + | IP-10 | MIG |
|---|---|---|---|---|---|
| | Cohort | Ki67 + % | Ki67 + 30% | Geometric mean | |
| Cohort | (mg/kg) | Mean change | | of fold-change | |
| 1 | 0.15 | 5.6 (n = 2) | 1.8 (n = 2) | 1.3 (n = 3) | 1.8 (n = 3) |
| 2 | 0.3 | 1.7 (n = 3) | 1.6 (n = 3) | 1.0 (n = 3) | 1.2 (n = 3) |
| 3 | 1 | 8.9 (n = 5) | 5.2 (n = 5) | 1.4 (n = 6) | 2.0 (n = 6) |
| 4 | 3 | 16.0 (n = 5) | 9.1 (n = 5) | 2.6 (n = 6) | 2.8 (n = 6) |
| 5 | 6 | 13.6 (n = 7) | 6.8 (n = 7) | 3.5 (n = 8) | 3.2 (n = 8) |
| 6 | 10 | 21.4 (n = 7) | 11.5 (n = 7) | 2.7 (n = 7) | 3.8 (n = 7) |

Conclusions: XmAb20717 was generally safe and has demonstrated PD activity in heavily pretreated patients with selected advanced solid tumors.

### Example 2

The activity of XmAb20717 was determined in an in vitro assay measuring IL-2 secretion from human lymphocytes stimulated with SEB, a method of assessing in vitro activity that has been used for other checkpoint inhibitors, including nivolumab. SEB-stimulated PBMC were treated with XmAb20717 or comparators/controls for 24 hours, and lymphocyte function was determined by measuring by ELISA the amount of IL-2 in culture supernatants.

When compared to an anti-RSV isotype-control bivalent antibody (XENP15074), XmAb20717 promoted a 4.1-fold increase in IL-2 secretion while XENP16432, a benchmark anti-PD1 bivalent antibody derived from the Fv of nivolumab with substitutions in the Fc domain similar to those in the XmAb20717 Fc domain, promoted a 2.6-fold increase in IL-2 secretion versus control. When compared to PD1 blockade by a bivalent PD1 antibody, XmAb20717 promoted a 1.5-fold increase in IL-2 secretion.

To determine if the avidity arising from XmAb20717's simultaneous binding to PD1 and CTLA4 contributed to IL-2 secretion, the intact bispecific antibody was compared to a mixture of its monovalent and monospecific component antibodies. Compared to a mixture of XENP20111 (a monovalent anti-PD1 scFv-Fc component antibody of XmAb20717) and XENP20059 (a monovalent anti-CTLA4 Fab-Fc component antibody of XmAb20717), XmAb20717 promoted a 1.6-fold increase in IL-2 secretion, suggesting that the increased avidity of XmAb20717 for dual-positive PD1 and CTLA4-expressing T cells contributes to its observed in vitro.

What is claimed is:

1. A method for treating a solid cancerous tumor in a human subject, comprising:
administering to the human subject having the solid cancerous tumor an intravenous dose, once every 13-15 days, of between about 0.05 mg/kg and about 12 mg/kg of a bispecific antibody comprising a first monomer comprising SEQ ID NO: 1, a second monomer comprising SEQ ID NO: 2, and a light chain comprising SEQ ID NO: 3 for a time period sufficient to treat the solid cancerous tumor.

2. The method of claim 1, wherein the solid cancerous tumor is selected from the group consisting of melanoma, triple negative breast cancer, hepatocellular carcinoma, urothelial carcinoma, squamous cell carcinoma of the head and neck, clear cell predominant type renal cell carcinoma, high microsatellite instability colorectal carcinoma, mismatch repair deficient colorectal carcinoma, endometrial carcinoma, small cell lung carcinoma, non-small cell lung carcinoma, gastric adenocarcinoma, and gastroesophageal junction adenocarcinoma.

3. The method of claim 1, wherein the solid cancerous tumor is melanoma, and the melanoma is cervical carcinoma.

4. The method of a preceding claim, wherein the intravenous dose is:
between about 0.05 mg/kg and about 0.25 mg/kg; or
between about 0.2 mg/kg and about 0.4 mg/kg; or
between about 0.5 mg/kg and about 1.5 mg/kg; or
between about 2.0 mg/kg and about 4.0 mg/kg; or
between about 8.0 mg/kg and about 12.0 mg/kg.

5. The method of a preceding claim, wherein the intravenous dose is between about 0.15 mg/kg and about 10.0 mg/kg.

6. The method of a preceding claim, wherein the administering of the intravenous dose to the human subject is between about 45 minutes and about 75 minutes.

7. The method of a preceding claim, wherein the time period sufficient to treat the cancer is between about 3 weeks and about 9 weeks.

8. The method of a preceding claim, further comprising, prior to the administering, assessing the weight of the human subject.
